(19) United States
(12) Patent Application Publication
(10) Pub. No.: US 2013/0316333 A1
(43) Pub. Date: Nov. 28, 2013

(54) Method for Detecting and Quantifying Microorganisms

(71) Applicants: Yoann Roupioz, Theys (FR); Roberto Calemczuk, Grenoble (FR); Thierry Vernet, Grenoble (FR); Thierry Livache, Jarrie (FR); Sihem Bouguelia, Montpellier (FR); Claire Durmort, Quaix En Chartreuse (FR)

(72) Inventors: Yoann Roupioz, Theys (FR); Roberto Calemczuk, Grenoble (FR); Thierry Vernet, Grenoble (FR); Thierry Livache, Jarrie (FR); Sihem Bouguelia, Montpellier (FR); Claire Durmort, Quaix En Chartreuse (FR)

(73) Assignee: Commissariat A L'Energie Atomique Et Aux Energies Alternatives, Paris (FR)

(21) Appl. No.: 13/990,992

(22) PCT Filed: Nov. 30, 2011
(86) PCT No.: PCT/IB11/55384

§ 371 Date: Aug. 13, 2013

(30) Foreign Application Priority Data
Dec. 1, 2010 (FR) 10 59983

(51) Int. Cl. C12Q 1/04

(52) U.S. Cl. CPC C12Q 1/04
USPC 435/5; 435/7.2; 435/6.15; 435/287.1; 435/7.32; 435/7.31; 435/287.2

(57) ABSTRACT

The present invention relates to a method for detecting at least one microorganism in a sample, including: cultivating the sample in a liquid medium in the presence of at least one specific ligand of the microorganism, and at least one scavenger having a lower affinity to the microorganism than the ligand, the binding of a compound to the ligand producing a first measurable signal and the binding of a compound to the scavenger producing a second measurable signal; determining the values of the first and second signals for at least one cultivation period; wherein it is deduced that the sample includes the microorganism when the values of the first signal and second signal are different for the same cultivation period.

### FIELD OF THE INVENTION

The present invention relates to a method for detecting and quantifying microorganisms in a sample.

### BACKGROUND OF THE INVENTION

The detection of live microorganisms in certain samples is crucially important, in particular in the medical field and the food-processing industry.

The standard method of detection remains microbial culture which often requires several days to obtain a sufficient number of microorganisms to identify the microorganism being tested for. In the case of testing using a sample derived from a patient, this delay before obtaining results often means that a broad-spectrum preventive antibiotic has to be administered to the patient. In the case of pathogenic bacteria, an increase in the number of antibiotic-resistant strains is therefore a possible consequence of this medication protocol which does not target a particular bacterium, but a multitude of other bacterial species.

Bacteriological diagnosis is conventionally carried out in two steps:
a first phase of growing the bacteria, generally on a solid medium, so as to have a sufficient number thereof and to isolate the suspect bacterium; at this stage, a study, on a solid culture medium, of the shape of the colonies allows a preliminary identification of the bacteria; in addition, this phase is frequently coupled with nonspecific detection of the bacteria, by culturing in a liquid medium, based on acidification of the culture medium or variations in the optical properties of the medium, for example by light scattering, which gives an idea as to the possible presence of bacteria in the sample;
a second phase of identifying the bacterium by growth in a selective medium, and then of studying the biochemical and immunological characteristics, according to a dichotomous key, ranging from the most vast characteristics to the most precise, so as to result in a particular bacterial species.

In medical applications, an additional step before the therapeutic treatment is often necessary; this consists in performing an antibiogram to test the level of resistance of the bacterium to the various antibiotics in the pharmacopeia.

This method requires at least 24 h of growth and, for certain bacterial species, up to several days of culture prior to visual identification. In any event, the length of time required for the analysis is crucial, and the enrichment and analysis steps are very lengthy (one to several days).

In order to reduce this delay, faster in vitro methods for detecting bacterial growth have been developed. However, all these approaches are based on multi-step systems involving, first, a culture phase in order to increase the number of bacteria present in the sample and then at least one identification and characterization phase.

Automated devices for monitoring bacterial growth have recently been introduced onto the market, for example Bactec 9000 from Becton-Dickinson and BacT/Alter from BioMérieux. These automated devices work on the basis of the assaying of CO₂ given off during bacterial proliferation. The use of these automated devices for blood culture has made it possible to increase test sensitivity, in particular during blood culture, and consequently to reduce response times (Lamy et al. (2005) Biotribune 16:37-39; Croizet et al. (2007) Spectra Biologie 160:45-51). However, these automated devices do not make it possible to specifically identify the bacterial genus and species.

More recently, the company Accelr8 has launched a microfluidic concentration device which nonspecifically adsorbs bacteria onto a chemically functionalized polymer surface. In this case, detection of microorganism growth precedes the identification of said microorganisms. Indeed, the bacteria are first multiplied on a support and then identified by virtue of an immunofluorescence method followed by an analysis by microscopy. It should be noted that this method is still relatively laborious since the two steps of culturing and then detection are carried out successively.

Fairly similar methods involving a system of electrical pulses for concentrating Bacillus subtilis spores in order to detect them using an associated optical system have been developed by Zourob et al. (2005) Lab Chip 5:1350-1365. This method does not involve any culturing, and directly detects the spores present in a sample. However, it requires the use of fairly complex microsystems.

Techniques using electrochemical detection methods have also been proposed. The most effective are based on impedimetric principles (Yang et al. (2004) Anal. Chem. 76:1107-13; Huang et al. (2010) Biosensors & Bioelectronics 25:1204-11). These methods often involve the use of a redox mediator used as a probe. In this case, the assaying medium has to be supplemented with the mediator, typically hexa-cyanoferrate, used at concentrations of from about 1 to 10 mM. This remains a major drawback since the effect of relatively high concentrations (mM) of compounds of this type on the microorganisms to be detected is still not properly understood and may be a source of interference with the biological medium.

Tracer-free optical techniques can in principle dispense with the addition of exogenous compounds and are therefore more direct. The surface plasmon resonance (SPR) technique has been applied to the detection of bacterial lysates by Taylor et al. (2006) Biosensors and Bioelectronics 22:752-758. In this case, detection is preceded by bacterial lysis and then the signal is amplified by binding with a specific secondary antibody. Limits of detection of between 10⁴ and 10⁶ bacteria/ml are achieved. This method therefore requires either highly contaminated samples or prior culturing in order to achieve these thresholds. Furthermore, given that detection is carried out on bacterial lysates, the method is not suitable for identifying live bacteria.

Finally, other studies have been reported that make it possible to detect the germination of Aspergillus niger and Candida albicans spores using micro-cantilevers (Nugaeva et al. (2007) Microsc. Microanal. 13:13-17; Nugaeva et al. (2005) Biosensors and Bioelectronics 21:849-856). This type of detection is carried out in air, in a humid chamber, and has the drawback that is cannot be carried out in a liquid medium. Moreover, it is only applicable to the detection of the germination of previously captured fungal spores.

### SUMMARY OF THE INVENTION

The present invention follows from the discovery, by the inventors, that coupling the culturing of a microorganism and the measuring of a differential signal generated by the specific binding of the microorganism by a ligand, i.e. the culturing and the measuring of the differential signal are carried out simultaneously, made it possible to lower the threshold of detection of the microorganism and to significantly reduce the analysis time, compared with the usual techniques.

Consequently, the present invention relates to a method for detecting at least one microorganism present in a sample, comprising:
culturing the sample in a liquid medium in the presence of at least one ligand specific for the microorganism and at least one sensor having a lower affinity for the microorganism than that of the ligand, the binding of a compound to the ligand producing a first measurable signal and the binding of a compound to the sensor producing a second measurable signal;
determining values of the first and second signals for at least one culture time;
   wherein it is deduced that the sample comprises the microorganism when the values of the first signal and of the second signal are different for the same culture time.

In a particular embodiment of the method according to the invention, when the sample comprises a microorganism, the amount of microorganisms present in the sample at the beginning of the culture is also determined from the change in the value of the first signal as a function of the culture time.

In another embodiment of the method according to the invention, when the sample comprises a microorganism, the sensitivity of the microorganism to at least one antimicrobial is determined from the change in the value of the first signal as a function of the culture time after introduction of the antimicrobial into the culture.

The present invention also relates to a device suitable for carrying out a method as defined above, comprising at least one chamber suitable for culturing a microorganism in a liquid medium, the chamber comprising at least one ligand specific for the microorganism and at least one sensor which has no affinity for the microorganism, the binding of a compound to the ligand producing a first measurable signal and the binding of a compound to the sensor producing a second measurable signal, the ligand and the sensor being attached to a support so as to be in contact with the liquid medium to be studied.

### DETAILED DESCRIPTION OF THE INVENTION

As understood herein, a microorganism is preferably a single-cell or multicellular prokaryotic or eukaryotic organism. However, when the microorganism is multicellular, the cells constituting the multicellular microorganism are preferably homogeneous in terms of differentiation, i.e. the microorganism does not comprise cells having different specializations. Preferably, the microorganism according to the invention is a live microorganism, i.e. it is capable of multiplying. Moreover, the microorganism according to the invention is preferably a single-cell microorganism. In this respect, the microorganism according to the invention may be a single-cell form of a multicellular organism according to its stage of development or reproduction. In addition, a microorganism according to the invention may also consist of isolated cells or of fragments of isolated tissues of a metazoan. Thus, the microorganism according to the invention may also be a mammalian cell, in particular a human cell, such as a tumor cell or a blood cell, for example a lymphocyte or a peripheral blood mononuclear cell. Preferably, the microorganism according to the invention is therefore a microorganism, in particular a single-cell microorganism, selected from the group consisting of a bacterium, a fungus, a yeast, an alga, a protozoan, and an isolated cell of a metazoan. Particularly preferably, the microorganism according to the invention is a bacterium, in particular of the Streptococcus or Escherichia genus.

The sample according to the invention may be of any type, insofar as it can comprise a microorganism. Preferably, the sample according to the invention is selected from the group consisting of a biological sample, a food sample, a water sample, in particular a wastewater, fresh water or sea water sample, a soil sample, a sludge sample or an air sample. The biological samples according to the invention originate from live organisms or organisms that were alive, in particular of animals or plants. The samples originating from animals, in particular from mammals, may be liquid or solid, and comprise in particular blood, plasma, serum, cerebrospinal fluid, urine, feces, synovial fluid, sperm, vaginal secretions, oral secretions, respiratory specimens, originating in particular from the lungs, the nose or the throat, pus, ascites fluids, or specimens of cutaneous or conjunctival serosites. Preferably, the food samples according to the invention originate in particular from foods which may be raw, cooked or prepared, from food ingredients, from spices or from pre-prepared meals. Also preferably, the sample according to the invention is not purified or concentrated before being placed in culture according to the invention.

As will become clearly apparent to those skilled in the art, the culturing in a liquid medium according to the invention is carried out so as to obtain a multiplication of the microorganism possibly present in the sample placed in culture and which is intended to be detected. The techniques and conditions for culturing microorganisms in a liquid medium are well known to those skilled in the art, who know in particular how to define, for each microorganism, the suitable nutritive medium, the optimal growth temperature, for example 37° C. for many bacteria pathogenic to mammals, and also the atmosphere required for the multiplication of a microorganism according to the invention. Moreover, weakly selective culture media, i.e. those which allow the growth of a large number of microorganisms of different types, will be preferentially used. The culture time can also be adapted for each microorganism, according to its growth rate and its generation time, i.e. the time required for the microorganism to divide into two daughter microorganisms. Preferably, the culture time according to the invention is greater than or equal to the time required for the production of at least two successive generations of daughter microorganisms, which therefore enables at least one quadrupling, i.e. a 4-fold multiplication, of the number of microorganisms to be detected. Those skilled in the art will easily understand that it is not necessary to actually observe the quadrupling of the number of microorganisms in culture, since the microorganism to be detected may be absent from the sample according to the invention, but the culture time corresponds at least to that for obtaining a quadrupling of the number of microorganisms in a culture which actually contains the microorganism and which is carried out under the same conditions as those of the method of the invention. Thus, it is also preferred for the culture time according to the invention to be at least equal to twice the generation time or the doubling time of the microorganism to be detected.

As those skilled in the art will well understand, the generation or doubling time according to the invention is that which can be obtained under the culture conditions according to the invention. Generally, the culture time will be less than 72 h, 48 h or 24 h. In addition, the culturing may be stopped as soon as it has been possible to obtain the information being sought, namely detection, determination of the amount, or sensitivity to antimicrobials.

Advantageously, the method of the invention is sensitive and makes it possible to detect the presence of micro-organisms in samples containing low concentrations of microorganism, for example equal to 1 or less than 10, 10² or 10³ microorganisms/ml, which are usually undetectable directly, in particular using a biosensor. Thus, the method of the invention is preferably applied to samples which are suspected of containing a microorganism to be detected at a concentration of less than or equal to 10⁶, 10⁵, 10⁴, 10³, 10² or 10 microorganisms/ml, or equal to 1 micro-organism/ml.

The ligand according to the invention is of any type which makes it possible to specifically bind to a microorganism or to several related microorganisms. It may in particular be:
a natural receptor for the microorganism, such as a polysaccharide sugar or a complex lipid, i.e. a lipid comprising at least one nonlipid group, in particular of a protein, carbohydrate, phosphorus-containing or nitrogen-containing type; a protein or a glycoprotein, such as plasminogen, for example, which binds to several bacterial species of the Streptococcus genus; whole bacteriophages or viruses, which are optionally inactivated, bacteriophage or virus fragments or bacteriophage or virus proteins;
immunoglobulins, such as antibodies and fragments thereof comprising the variable part, specifically targeting an antigen, or the constant part, which can be bound by bacteria of the Staphylococcus genus which comprise protein A, or T-cell receptor (TCR) fragments, comprising in particular the variable part;
synthetic compounds, in particular:
   small organic molecules comprising in particular from 1 to 100 carbon atoms,
   compounds of peptide nature, such as scFvs (single chain variable fragments),
   compounds of polynucleotide nature, such as aptamers, or
   compounds of polysaccharide nature;
ground materials, lysates or extracts of live organisms or tissues;
synthetic materials which have a microorganism-adsorbing surface; or
mixtures comprising two or more of the ligands defined above.

Thus, the ligand according to the invention is preferably selected from the group consisting of an antibody, an antibody fragment, an scFv, an aptamer, a protein or a glycoprotein, whole bacteriophages, which are optionally inactivated, bacteriophage fragments, and bacteriophage proteins.

The sensor according to the invention acts as a negative control. Consequently, it is preferably of a nature similar to that of the ligand. Moreover, the sensor according to the invention has less affinity for the microorganism to be detected than that of the ligand, i.e., according to the invention, it preferably has an affinity for the microorganism at least 10 times lower, in particular at least 100 times, 1000 times, 10 000 times, 100 000 or 1 000 000 times lower than that of the ligand for the microorganism. In particular the affinities of the ligand and of the sensor for the microorganism can be determined by the Scatchard method under the same experimental conditions. More preferably, the sensor according to the invention has no affinity for the microorganism.

Preferably, it is considered according to the invention that the values of the first signal and of the second signal for the same culture time are different when the value of the difference between the first signal and the second signal is greater than or equal to twice the measured background noise signal. The measured background noise signal corresponds in particular to the first signal measured in the absence of microorganism.

As understood herein, the measurable signal according to the invention is directly generated by the attachment or the binding of a compound, in particular a microorganism, to the ligand or the sensor.

In particular, the measurable signal according to the invention preferably does not come from a mediator, such as an oxidation-reduction probe, or from a marker present in the medium, or added to the medium, other than the ligand and sensor according to the invention. Thus, preferably, the measurable signal according to the invention does not come from the additional attachment of a marker specific for the microorganism to a microorganism already bound by the ligand or the sensor.

The signal may be measured by any technique suitable for measuring at least two signals simultaneously, and which is in particular direct, and especially by microscopy, by surface plasmon resonance, by resonant mirrors, by impedance measurement, by a microelectromechanical system (MEMS), such as quartz microbalances or flexible beams, by measurement of light, in particular ultraviolet or visible light, absorption, or else by measurement of fluorescence, in particular if the microorganism is itself fluorescent, which are well known to those skilled in the art. In this respect, as will become clearly apparent to those skilled in the art, the marker defined above does not denote a microorganism according to the invention; thus, the signal according to the invention may come from the microorganism when it is bound by the ligand or the sensor. Surface plasmon resonance is particularly preferred according to the invention.

Preferably, the ligand and the sensor are attached to a support. More preferably, the support enables the transduction of the signal produced by the binding of a compound to the ligand and/or to the sensor, in particular such that the signal can be measured by the techniques mentioned above. Such supports are well known to those skilled in the art and comprise, in particular, transparent substrates covered with continuous or discontinuous metal surfaces, suitable for measuring by surface plasmon resonance. Thus, in a preferred embodiment of the invention, the ligand and the sensor are attached to a support, identical or different, which is itself constitutive of a biochip. Moreover, the ligand and the sensor, or the support(s), or the device, according to the invention can be comprised in a container intended for collecting liquids which may contain microorganisms, or intended for culturing microorganisms, such as a blood culture flask or a Stomacher bag, for example.

Also preferably, the signal is measured in real time, in particular without it being necessary to take samples of the culture in order to measure the signal. The measured signal is preferably recorded, for example in the form of a curve showing the intensity of the measured signal as a function of time. It is also possible to record images of the support to which the ligand and the sensor according to the invention are attached, in order to determine the degree, or the level, of occupation of the ligand and of the sensor by the microorganism.

In particular embodiments of the invention, the method according to the invention makes it possible to detect the presence of several different microorganisms, to determine the amount of various microorganisms present in the sample, or to determine the sensitivity of various microorganisms to one or more antimicrobials; several ligands respectively specific for the various microorganisms need then be used. Such a method is then said to be a multiplex method.

As understood herein, the term “antimicrobial” refers to any microbicidal compound which inhibits the growth of the microorganism or which reduces its proliferation, in particular to any antibiotic, bactericidal or bacterio-static compound, such as erythromycin. Moreover, the term “antimicrobial” also refers, in particular when the microorganism according to the invention is a tumor cell, to any anticancer, in particular chemotherapy, compound intended to destroy the microorganism or to reduce its proliferation.

Preferably, when the sensitivity of the microorganism to at least one antimicrobial is determined according to the invention, the change in the value of the first signal as a function of the culture time originates, partly or totally, from a variation in the time required for the division of a microorganism, i.e. the generation time, and thus from the change over time in the number of microorganisms which bind to the first ligand. As those skilled in the art will well understand, if the generation time of the microorganism increases significantly, the variation in the number of microorganisms will be smaller than that obtained in the absence of the antimicrobial. Thus, preferably, the change in the value of the first signal is not due to the destruction by the antimicrobial of the microorganisms attached to the first ligand according to the invention.

### DESCRIPTION OF THE FIGURES

FIGS. 1, 2 and 3

FIGS. 1 to 3 stem from three independent experiments and represent the variation in reflectivity (dR, y-axis, as %) measured by surface plasmon resonance imaging, as a function of the culture time (x-axis, time in min), of a biochip functionalized using (i) anti-CbpE antibodies (large stars, triplicate) directed against Streptococcus pneumoniae, (ii) plasminogen (Plg) (large circles, triplicate), (iii) IgG not specific for S. pneumoniae (large triangles, triplicate) and (iv) pyrrole only (large squares), and placed in the presence of a culture of S. pneumoniae strain R6 at the initial concentration of 10² bacteria/ml (FIG. 1), 10³ bacteria/ml (FIGS. 2) and 10⁴ bacteria/ml (FIG. 3), in a sample volume of 500 μl (i.e., respectively, 50, 500 and 5000 bacteria). Moreover, the curves obtained using the same biochip placed in the presence of a noninoculated culture medium are also presented, as controls, and denoted (c).

FIG. 4

FIG. 4 represents the variation in reflectivity (ΔR_{SPR}, y-axis, as %), measured by surface plasmon imaging, of a biochip functionalized using anti-CbpE antibodies directed against Streptococcus pneumoniae (positive spot) and pyrrole only (negative spot), in the presence of a culture of S. pneumoniae, as a function of the culture time (x-axis, t in min), and also a curve modeling the reflectivity of the spot functionalized using anti-CbpE antibodies (calculated curve).

FIG. 5

FIG. 5 represents the variation in reflectivity (dR, y-axis, as %) of a biochip functionalized using anti-CbpE antibodies directed against Streptococcus pneumoniae, plasminogen (Plg), IgG not specific for S. pneumoniae, and pyrrole only, placed in the presence of a culture of S. pneumoniae strain R6 at the initial concentration of 10⁴ bacteria/ml in two vessels in parallel, to which are respectively added, at time t=250 min, (i) erythromycin at a final concentration of 40 mg/ml diluted in ethanol at a final concentration of 0.08% (+ATB), (ii) ethanol (EtOH) at a final concentration of 0.08%.

### EXAMPLES

### Example 1

### Detection of Microorganisms in a Sample

### Construction of a Biochip

A protein biochip was prepared using the method of electropolymerization of proteins on a gold-coated prism (used as a working electrode), as described by Grosjean et al. (2005) Analytical Biochemistry 347:193-200, using a protein-pyrrole conjugate in the presence of free pyrrole. Briefly, the electropolymerization of the free pyrrole and of the proteins coupled to pyrrole-NHS is carried out with a pipette tip containing a platinum rod acting as a counterelectrode; the polymerization is carried out by means of rapid electrical pulses of 100 ms (2.4 V) between the working electrode and the counterelectrode, as is in particular described by Guédon et al. (2000) Anal. Chem. 72:6003-6009. Each protein entity was deposited in triplicate on the gold-coated surface of the prism in order to estimate the reproducibility of the process.

The ligands used are the following:
Ligands which recognize Streptococcus pneumoniae
   (i) anti-CbpE antibodies prepared according to Attali et al. (2008) Infect. Immuno. 76:466-76;
   (ii) human plasminogen (Sigma Aldrich).
Sensors for the negative controls:
   (iii)purified rabbit IgG (Sigma Aldrich) or anti-botulinum toxin monoclonal IgG;
   (iv) pyrrole (Tokyo kasei, TCI).

All these products were coupled to pyrrole and then deposited in the form of spots on the gold-coated prism according to a deposit plan suitable for the shape of the vessels for culturing the microorganisms. Each vessel is provided with 2 independent compartments which can contain two different samples, one of which is a control.

### Preparation of Bacterial Cultures

The R6-strain pneumococci were cultured in Todd Hewitt culture medium (TH from Mast Diagnostic). The culture was stopped during the optimum growth phase of the bacteria, i.e. at an optical density measured at 600 nm (OD₆₀₀) of 0.4, which corresponds to a bacterial concentration of approximately 10⁸ bacteria/ml. Successive dilutions were carried in TH medium in order to obtain concentrations of 10² to 10⁴ bacteria/ml.

### Measurement by Surface Plasmon Resonance Imaging (SPRi):

The measurements by SPR imaging were carried out using the SPRi-Lab system from Genoptics (Orsay, France).

The biochip was preblocked with PBS buffer comprising 1% bovine serum albumin (BSA) for 15 minutes. 0.5 milliliter of sample possibly containing bacteria is placed in the “sample” compartment, while culture medium devoid of pneumococci is placed in the “control” compartment. The system is placed in the chamber of the SPRi instrument heated to 37° C. The vessel is stoppered in order to prevent evaporation of the culture medium during the growth carried out in the absence of agitation.

In parallel, growth controls were carried out. Several tubes containing 1 ml of the culture at 10³ bacteria/ml were prepared and incubated in an incubator at 37° C. The OD₆₀₀ was measured every hour, in order to monitor the change in the bacterial growth as a function of time and to compare it to the SPRi growth curves. The bacteria were cultured for 16 hours.

TABLE 1
Monitoring of growth by measuring optical density at 600 nm

| Culture time | OD |
|---|---|
| 1 hour | 0.08 |
| 2 hours | 0.13 |
| 3 hours | 0.15 |
| 4 hours | 0.17 |
| 5 hours | 0.19 |
| 6 hours | 0.25 |
| 7 hours | 0.46 |

### Study of Selectivity

After the injection of 500 bacteria, it is observed in FIG. 2 that the first signals, linked to the capture of bacteria by the anti-CbpE antibodies and also by plasminogen, are detectable after approximately 300 minutes. Beyond 400 minutes, a signal becomes visible for the spots bearing the negative controls (nonspecific attachments). The signals (c) obtained in the control vessel, without bacterial inoculation, remain negative.

Significant bacterial growth is also observed between 300 and 400 minutes on the basis of Table 1.

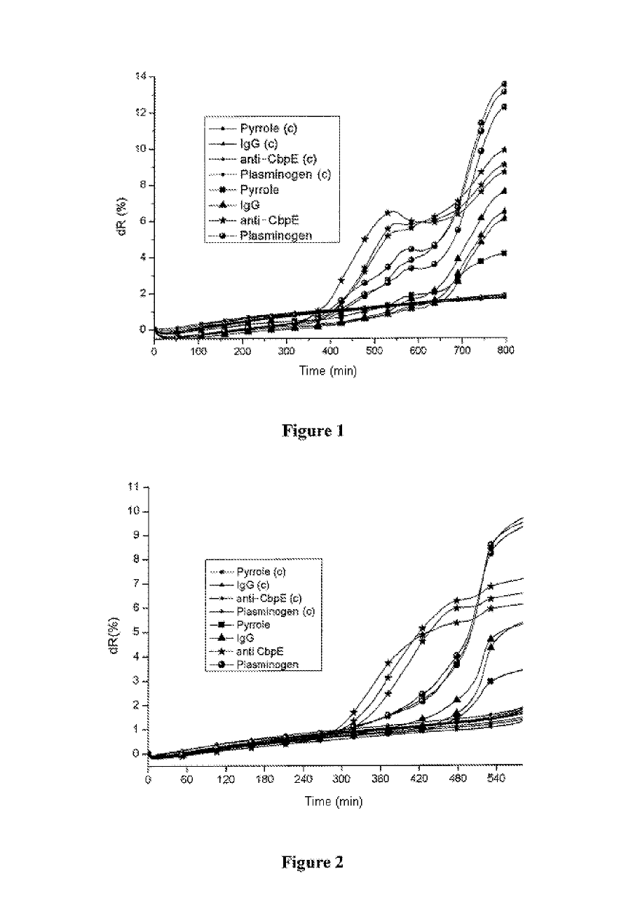

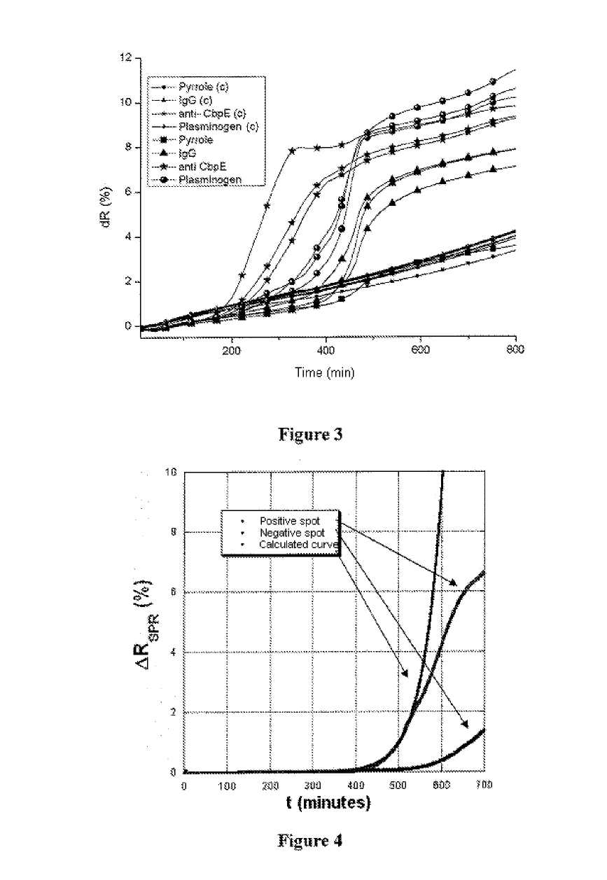

The injection of a smaller amount of bacteria (50 bacteria, FIG. 1), and of a larger amount (500 bacteria, FIG. 3), also generates an increase in visible signal. The time lag observed clearly correspond to a generation time of about 30 minutes.

### Example 2

### Quantification of the Bacteria Present in the Starting Sample

FIG. 4 shows the change over time of the variations in reflectivity measured by SPR imaging (ΔR_{SPR}), observed on the spot functionalized with the anti-CbpE antibody and on a negative control spot comprising only pyrrole.

It can be seen that, after a lag period of approximately 400 minutes, during which the change in the signal is masked by the experimental noise, the increase in reflectivity is clearly exponential.

In this respect, FIG. 4 also shows the curve representative of the function ΔR_{SPR}=Rₒ 2^{t/τ)}−Rₒ which models the change in ΔR_{SPR} observed on the spot functionalized with the anti-CbpE antibody using the value of 30 minutes for τ which is typical of the population doubling time associated with Streptococcus pneumoniae. The multiplication factor Rₒ is proportional to the number of microorganisms initially present in the sample. The determination of this factor therefore enables a quantitative evaluation of the bacteria initially present in the sample.

From 550 minutes, there is departure from this exponential system. This reduction in growth rate can be attributed to the limitations generated by modifications of the culture medium, in particular the depletion thereof liable to significantly affect the bacterial growth, as those skilled in the art are aware.

The negative control curve remains close to zero up to 600 minutes. The beginning of an increase in ΔR_{SPR}, attributable to the control nonspecific interactions between Streptococcus pneumoniae and the surface of the spot, can subsequently be observed.

### Example 3

### Inhibition of Growth by Adding Antibiotic

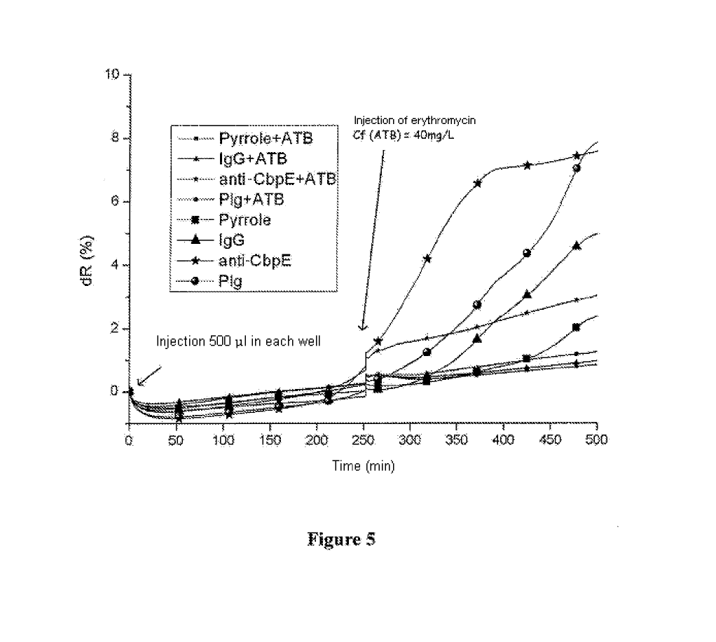

FIG. 5 shows the impact of the addition of an antibiotic (ABT) (erythromycin, Aldrich) which targets pneumococci, at a final concentration of 40 mg/ml, and of ethanol (EtOH) at a final concentration of 0.08%, on the reflectivity of a Streptococcus pneumoniae culture, inoculated at 10³ bacteria/ml, after 250 min. of culture.

It is observed that the addition of the antibiotic causes a clear decrease in the slope of the curves showing the reflectivity which is particularly marked for the spots bearing the anti-CbpE antibodies and the plasminogen. The decrease is therefore linked to an inhibition of bacterial growth. Moreover, no decrease is observed when control solution is added at the same ethanol concentration, thereby demonstrating that the inhibition of bacterial growth previously observed is directly attributable to the action of the antibiotic and not to a solvent effect.

Consequently, the method for quantifying bacterial growth of the invention is of use for establishing an antibiogram.

1. A method for detecting at least one microorganism present in a sample, comprising:
culturing the sample in a liquid medium in the presence of at least one ligand specific for the microorganism and at least one sensor having a lower affinity for the microorganism than that of the ligand, the binding of a compound to the ligand producing a first measurable signal and the binding of a compound to the sensor producing a second measurable signal;
determining values of the first and second signals for at least one culture time; wherein it is deduced that the sample comprises the microorganism when the values of the first signal and of the second signal are different for the same culture time.

2. The detection method as claimed in claim 1, wherein the culture time is at least twice the generation time of the microorganism.

3. The detection method as claimed in claim 1, wherein the microorganism is selected from the group consisting of a bacterium, a fungus, an alga, a protozoan, and an isolated cell of a metazoan.

4. The method as claimed in claim 1, wherein the ligand is selected from the group consisting of an antibody, an antibody fragment, an scFv, an aptamer (DNA, RNA), a protein or a glycoprotein, whole bacteriophages or viruses, which are optionally inactivated, bacteriophage or virus fragments, and bacteriophage or virus proteins.

5. The method as claimed in claim 1, wherein the sample is selected from the group consisting of a biological sample, a food sample, a water sample, a soil sample and an air sample.

6. The method as claimed in claim 1, wherein the ligand and/or the sensor are attached to a support for transduction of the signal produced by the binding of a compound to the ligand and/or to the sensor.

7. The method as claimed in claim 1, wherein the first and second signals are measured in real time.

8. The method as claimed in claim 1, wherein the first or second signal is measured by microscopy, by surface plasmon resonance, by resonant mirror, by impedance measurement, by quartz microbalance, by flexible beam, by measurement of light absorption, or by measurement of fluorescence of fluorescent microorganisms.

9. The method as claimed in claim 1, wherein the first or second signal is measured in real time by surface plasmon resonance.

10. The method as claimed in claim 1, wherein the measurable first or second signal according to the invention does not come from a mediator or from a marker present in the medium, or added to the medium, other than the ligand and the sensor according to the invention.

11. The method as claimed in claim 1, wherein, when the sample comprises a microorganism, the amount of microorganisms present in the sample at the beginning of the culture is also determined from the change in the value of the first signal as a function of the culture time.

12. The method as claimed in claim 1, wherein, when the sample comprises a microorganism, the sensitivity of the microorganism to at least one antimicrobial is determined from the change in the value of the first signal as a function of the culture time after introduction of the antimicrobial into the culture.

13. The method as claimed in claim 1, wherein the presence or the amount of several different microorganisms in the sample is determined.

14. A device suitable for detecting at least one microorganism present in a sample, comprising at least one chamber suitable for culturing the microorganism in a liquid medium, the chamber comprising at least one ligand specific for the microorganism and at least one sensor having a lower affinity for the microorganism than that of the ligand, the binding of a compound to the ligand producing a first measurable signal and the binding of a compound to the sensor producing a second measurable signal, the ligand and the sensor being attached to a support so as to be in contact with the liquid culture medium.

15. The device as claimed in claim 14, further comprising a container intended for collecting liquids capable of containing microorganisms and/or for culturing microorganisms.
